(19) United States
(12) Patent Application Publication
(10) Pub. No.: US 2022/0287929 A1
(43) Pub. Date: Sep. 15, 2022

(54) SKIN CARE PRODUCT AND METHOD FOR ITS PRODUCTION

(71) Applicant: TheUbeauty.com, LLC, Jacksonville, FL (US)

(72) Inventors: Antonio Catalano, Padova (IT); Khalil Mitri, Padova (IT)

(73) Assignee: TheUbeauty.com, LLC, Jacksonville, FL (US)

(21) Appl. No.: 17/826,889

(22) Filed: May 27, 2022

Related U.S. Application Data
(63) Continuation-in-part of application No. PCT/US2020/061782, filed on Nov. 23, 2020.

(30) Foreign Application Priority Data
Dec. 3, 2019 (IT) 102019000022764

(51) Int. Cl. A61K 8/11; A61Q 19/00; A61K 8/92; A61K 8/67; A61K 8/36

(52) U.S. Cl. CPC A61K 8/11; A61Q 19/00; A61K 8/92; A61K 8/67; A61K 8/361; A61K 2800/522; A61K 2800/805

(57) ABSTRACT

This invention is a skin care product and method for making same. The skin care product includes at least one capsule, the capsule comprising a matrix, one or more active ingredients incorporated or contained in the matrix, where at least the matrix has a high affinity with Reactive Oxygen Species (ROS). In an embodiment of the invention, at least one antioxidant agent is incorporated in the matrix. A capsule is a lipid particle. In an embodiment of the invention, the skin care product comprising the capsules is used for topical inhibition of Reactive Oxygen Species ROS in the skin of a person. A method for making the capsule is also disclosed.

### CROSS-REFERENCE TO RELATED APPLICATIONS

Pursuant to 35 U.S.C. sections 111(a), 120, and 365, this application is a continuation-in-part of and claims the benefit of priority from co-pending PCT patent application serial no. PCT/US2020/061782 entitled “Skin Care Product and Method for its Production” filed on Nov. 23, 2020, which claims the benefit of priority from Italian patent application serial no. 102019000022764 entitled “Skin Care Product and Method for its Production” filed on Dec. 3, 2019; the entire disclosures of both are incorporated herein by reference.

### FIELD OF THE INVENTION

The invention relates generally to capsules, or lipid particles, particularly for use in skin care products and a method for making capsules and skin care products containing capsules.

### BACKGROUND OF THE INVENTION

In recent times, skin care products such as creams, lotions, or serums, composed of a fluid substance, usually a gel, that incorporates capsules containing one or more molecules of cosmetic active ingredients, have become widespread.

The term “active ingredient” refers to a substance that possesses a biological activity or that influences the appearance and condition of the skin. The term “biological activity” refers to the fact that the substance has an effect on a biological tissue, in this case the skin.

An example of an effect on a biological tissue is a cosmetic effect. Another example of an effect on a biological tissue, particularly on the skin, is a local reduction of free radicals.

Examples of active ingredients include vitamins, such as vitamin A, C, E, and their derivatives, coenzyme Q10, minerals, antioxidants, natural extracts, and peptides.

Some of these active ingredients have a notable affinity with Reactive Oxygen Species (ROS). The term “affinity” refers to the tendency of chemical elements and/or compounds to react together.

In particular, the affinity between active ingredients and ROS can be measured through the Oxygen Radical Absorbance Capacity (ORAC). The higher the ORAC number, the more sensitive the molecule measured is to ROS, that is, it has an affinity toward them.

The types of damage caused by an excess, or in any case an imbalance, in Reactive Oxygen Species (ROS) are well known, the most serious of which is the acceleration of aging of the skin. In particular, more damage is done to the skin in areas with the greatest concentration of ROS. ROS determines lipid peroxidation. Lipid peroxidation is a process that occurs due to “free radicals” containing molecular oxygen with an unpaired electron, or electron deficiency, (“peroxyl”).

Because of this, lipids containing unsaturated fatty acids and their esters are directly oxidized by the molecular oxygen and the damage is capable of propagating itself through a chain reaction, in which lipids with an unpaired electron tend to reintegrate the loss by taking electrons from contiguous molecules, until it also involves the proteins of the central nucleus and the DNA.

Some of the possible damage caused by these reactions are lipid peroxidation, amino acid and protein peroxidation, enzymatic alterations, and damage to protein structures.

The ROS generated within mitochondria can directly damage the mtDNA and other mitochondrial components.

The ROS can also damage nuclear DNA, which leads to the activation of the tumor suppressor.

For these reasons, the oxidative modification of some proteins can be an important factor in aging.

Products containing active ingredients, such as antioxidant active ingredients, capable of interacting with and inhibiting ROS activity are known and widespread. Examples of such products are Revitalift® Derm Intensives 10% Pure Vitamin C by L'Oreal Paris, Resveratrol B E Antioxidant Night concentrate by Skinceuticals, DayWear Advanced Multi-Protection Anti-Oxidant Crème SPF 15 by Estee Lauder, and Athelios AOX Daily Antioxidant Serum with Sunscreen by La Roche-Posay.

These well-known techniques have some drawbacks. Although known products as of today include active ingredients sensitive to ROS, they have the drawback of being incorporated into capsules with an oxidization-stable matrix, or containment casing, designed to protect the content or to convey it in a controlled and specific manner but not to have affinity towards ROS. This matrix generally comprises polymers and/or saturated lipids that are oxidization-stable or with a reduced polyunsaturated content. The term “stable matrix” refers to a matrix with an ORAC <500 μmol/100 g.

The expression “high affinity to ROS” refers to a matrix with an ORAC >500 μmol/100 g.

The matrices used in known products have the purpose of protecting the active ingredients incorporated and/or making them more bioavailable and/or increasing cutaneous penetration and/or gradual release. The term “bioavailable” means the substance is capable of entering the circulatory system.

For these reasons, however, the active ingredients are conveyed equally on all areas of the skin on which the product is applied regardless of the localized concentration of ROS and are not limited to the areas of greatest concentration of ROS, where they would be most needed.

Furthermore, the production of such products requires the use of organic solvents, such as dichloromethane, chloroform, diethyl ether and ether-methanol mixtures, which can be toxic, and/or high temperatures, higher than 60 degrees Celsius in the order of 100 degrees Celsius. This results in an economically and energetically expensive process.

Therefore, there exists a need to produce a skin care product and a procedure for its production capable of overcoming one or more drawbacks of known products and methods.

There also exists a need to produce a skin care product with capsules comprising a matrix that is highly sensitive to ROS and capable of conveying the active ingredients incorporated within it to areas with ROS, particularly those with the greatest concentration of ROS.

There also exists a need to produce a skin care product with capsules that have greater affinity to ROS than known products.

There also exists a need for a method for the production of a skin care product that does not require the use of organic solvents and high temperatures.

There also exists a need to produce a skin care product and method for its production that is highly reliable and relatively easy to implement, at a competitive cost.

Further, there exists a need to overcome the drawbacks of the known technique as an alternative to any existing solutions.

### BRIEF SUMMARY OF THE INVENTION

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. Further, features of the various disclosed embodiments of the invention can be combined and such combinations fall within the scope of the invention.

The invention includes a skin care product comprising at least one capsule, with this capsule comprising a matrix, one or more active ingredients embedded or contained in said matrix, said product being characterized in that at least said matrix has a high ROS affinity.

In the context of this invention a capsule is a lipid particle. A lipid particle is a vesicle or agglomerate primarily made of lipid components, usually in a gel or semi-solid shape, which may contain and deliver different molecules. Examples of lipid particles are liposomes, lipid nanoparticles, and micelles.

The invention further includes a method for the making of capsules to be incorporated into a skin care product. This method is characterized in that it comprises the following steps: melting a solid lipophilic part at approximately 50±5 degrees Celsius, obtaining a melted lipophilic part, solubilizing/dispersing in water: one or more water-soluble active ingredients, at least one lipophilic component rich in unsaturated fatty acids, at least one surfactant and/or suspension agent and/or humectant, at least one antioxidant agent, obtaining an aqueous solution or suspension, cooling said aqueous solution or suspension to a temperature lower than 10 degrees Celsius, obtaining a cooled aqueous solution or suspension, combining said melted lipophilic part with said cooled aqueous solution or suspension, under stable agitation, obtaining an emulsion, screening said emulsion through a mesh with holes with a size on the order of 0.1-10 μm, obtaining a microemulsion, composed of said at least one capsule, drying said microemulsion in a vacuum at a controlled temperature lower than 40 degrees Celsius and/or adding preservatives to the microemulsion.

The invention comprises a skin care product comprising one or more capsules in a matrix that is highly sensitive to ROS and capable of conveying the active ingredients incorporated within it to areas with ROS to skin, particularly those areas of skin with the greatest concentration of ROS.

In various embodiments of the invention, a method of the production of a skin care product does not require the use of organic solvents and high temperatures.

A skin care product of the invention comprises capsules with a greater affinity to ROS than prior art products.

Furthermore, this invention overcomes the drawbacks of the known technique as an alternative to any existing solutions.

A skin care product of the present invention and the disclosed methods for making same is highly reliable and relatively easy to implement, at a competitive cost.

The invention produces a skin care product with capsules having a greater affinity to ROS than similar products known as of today.

Furthermore, the invention refines a method for the production of a skin care product that does not require the use of organic solvents and high temperatures.

The includes a skin care product and method for its production that is highly reliable and relatively easy to implement, at a competitive cost.

In an embodiment, the invention comprises a capsule, comprising: a matrix comprising: a lipid component having a melting temperature from greater than or equal to 40 degrees Celsius to less than or equal to 55 degrees Celsius, wherein the lipid component is from greater than or equal to 20 percent to less than or equal to 60 percent by weight of the total weight of the capsule; an unsaturated fatty acid, wherein the unsaturated fatty acid is from greater than or equal to 20 percent to less than or equal to 30 percent by weight of the total weight of the capsule; an excipient wherein the excipient is from greater than or equal to 10 percent to less than or equal to 12 percent by weight of the total weight of the capsule; and an antioxidant agent incorporated in said matrix.

In an embodiment, the invention comprises a process for the preparation of a capsule, comprising the steps of: providing a lipid component having a melting temperature of greater than or equal to 40 degrees Celsius to less than or equal to 55 degrees Celsius; melting the lipid component to form a melted lipid component; dispersing an unsaturated fatty acid in the melted lipid component to form a dispersion of the lipid component and the unsaturated fatty acid; dispersing an antioxidant agent and an excipient in water previously heated to a temperature from greater than or equal to 40 degrees Celsius to less than or equal to 50 degrees Celsius to form a dispersion of water, antioxidant agent, and excipient; combining the dispersion of water, antioxidant agent, and excipient with the dispersion of the lipid component and the unsaturated fatty acid, under stirring, to form an emulsion; and cooling the emulsion to a temperature from greater than 5 degrees Celsius to less than 15 degrees Celsius, to form an emulsion of capsules in an aqueous phase.

In an embodiment, the invention comprises a method for topical treatment of a condition associated with an accumulation of Reactive Oxygen Species (ROS) in an area of skin of a person, comprising applying a capsule to the area of skin of the person, the capsule comprising: a matrix comprising: a lipid component having a melting temperature from greater than or equal to 40 degrees Celsius to less than or equal to 55 degrees Celsius, wherein the lipid component is from greater than or equal to 20 percent to less than or equal to 60 percent by weight of the total weight of the capsule; an unsaturated fatty acid, wherein the unsaturated fatty acid is from greater than or equal to 20 percent to less than or equal to 30 percent by weight of the total weight of the capsule; an excipient wherein the excipient is from greater than or equal to 10 percent to less than or equal to 12 percent by weight of the total weight of the capsule; and an antioxidant agent incorporated in said matrix.

### DESCRIPTION

The following is a detailed description of embodiments to illustrate the principles of the invention. The embodiments are provided to illustrate aspects of the invention, but the invention is not limited to any embodiment. The scope of the invention encompasses numerous alternatives, modifications, and equivalents. The scope of the invention is limited only by the claims.

While numerous specific details are set forth in the following description to provide a thorough understanding of the invention, the invention may be practiced according to the claims without some or all of these specific details.

References made to particular examples and implementations are for illustrative purposes and are not intended to limit the scope of the claims.

This invention is for one or more skin care products and methods for the making of one or more skin care products.

A skin care product is disclosed where the skin care product comprises at least one capsule with the capsule comprising a matrix, one or more active ingredients embedded or contained in said matrix, wherein at least said matrix has a high ROS affinity.

A capsule is a lipid particle. A lipid particle is a vesicle or agglomerate primarily made of lipid components which may contain and deliver different molecules. Examples of lipid particles include but are not limited to liposomes, lipid nanoparticles, and micelles.

A skin care product, according to the invention, comprising at least one capsule, each comprising a matrix and one or more active ingredients embedded or contained in said matrix.

In an embodiment of this invention the skin care products contain multiple capsules. The matrix has a high affinity to ROS.

In an embodiment, the matrix comprises up to 70% by weight of polyunsaturated fatty acids with a high affinity to ROS. In this way, the matrix increases the sensitivity of the capsules to ROS and breaks contact with them, releasing the active ingredients contained within the capsules.

Specifically, each of these capsules comprises a solid lipophilic part with a melting point above 50 degrees Celsius, with a percentage by weight between 0.1% and 10%, a lipophilic part of unsaturated fatty acids with a percentage between 10% and 70% by weight, surfactants and/or suspension agents and/or humectants, with a percentage by weight between 0.1% and 10%, active ingredients, with a percentage by weight between 0.01% and 20%.

This lipophilic solid part includes, in one example embodiment, Acacia decurrens and/or jojoba and/or sunflower seed wax and/or polyglycerol-3 esters.

This lipophilic part of unsaturated fatty acids includes at least one lipophilic component rich in unsaturated fatty acids such as, in one example embodiment, linoleic acid and/or linolenic acid and/or alpha linoleic acid and/or oleic acid. The term “rich in unsaturated fatty acids” means that it includes more than 10% fatty acids by weight.

Such surfactants and/or suspension agents and/or humectants are, for example, Polysorbate 20 and/or xanthan gum and/or glycerin.

These active ingredients are: at least one water-soluble active ingredient and at least one antioxidant agent.

The water-soluble active ingredients are, in various embodiments, vitamins, peptides, minerals, growth factors, antioxidants, or natural extracts.

The antioxidant agents are, in various embodiments, vitamin E, vitamin C, BHT (butylhydroxytoluene), BHA (butylhydroxyanisole), or bisulphites.

In an alternate embodiment, a skin care product comprises one or more capsules, each capsule comprising a matrix and at least one antioxidant agent incorporated in the matrix. In this embodiment, the matrix comprises a lipid component having a melting temperature from greater than or equal to 40 degrees Celsius to less than or equal to 55 degrees Celsius. The lipid component is from greater than or equal to 20% to less than or equal to 60% by weight of the total weight of the capsule.

Advantageously, the aforementioned melting temperature is closer to the temperature of the skin (37 degrees Celsius) than the melting temperatures of other waxes used to create capsules, such as microcrystalline wax, carnauba wax, and/or bees wax, that have a melting temperature above 65 degrees Celsius. The advantage of this being that the heat of the skin can therefore melt the particles, thus improving absorption of their contents.

The lipid component is a mixture of at least two of mimosa wax (Acacia decurrens), jojoba seed wax, sunflower seed wax, polyglycerol, pentaerythrityl distearate, and transesterification products of polyglycerol and at least one wax chosen from jojoba wax, mimosa wax and sunflower seed wax.

The matrix further comprises one or more unsaturated or polyunsaturated fatty acids. The one or more unsaturated or polyunsaturated fatty acids are from greater than or equal to 20% to less than or equal to 30% by weight of the total weight of the capsule. By having a high affinity towards ROS, the one or more unsaturated or polyunsaturated fatty acids provide the capsules of the invention with the property of interacting with ROS and releasing the active ingredients in a targeted manner. The capsules of the invention have a matrix with a high affinity towards ROS.

Affinity towards ROS refers to the tendency that the active ingredients or capsules that contain the active ingredients have to interact with ROS.

ROS affinity is defined in terms of iodine index, measured according to ASTM D1959-97.

As described above, the capsules commonly used today have an oxidation stable matrix.

The expression “oxidation stable matrix” refers to a matrix stable to oxidation and, in the case of a lipid matrix, this means an iodine index of less than 60 g Iodine/100 g, where the iodine index is measured according to ASTM D1959-97 standard.

In one embodiment, the matrix of the capsule according to the invention has an iodine index higher or equal to 60 g Iodine/100 g of matrix where the iodine index is measured according to the ASTM D1959-97 standard. Therefore, the matrix according to the invention can more easily undergo oxidation by ROS than other commonly used versions.

The matrix comprises one or more unsaturated fatty acids. In one embodiment of the invention, the one or more unsaturated fatty acids are selected from the group consisting of C16-C24 unsaturated fatty acids, such as linoleic acid, linolenic acid, alpha linolenic acid, oleic acid, and/or their mixtures.

The one or more unsaturated fatty acids of the matrix comprise at least 60% by weight of polyunsaturated fatty acids of the total weight of the unsaturated fatty acids.

The capsules of the invention comprise at least one antioxidant agent that incorporated in the matrix. Examples of an antioxidant agent are BHT (butylhydroxytoluene), vitamin E, vitamin C, BHA (butylhydroxyanisole), polyphenols, carotenoids, superoxide dismutase and bisulfites. The term “vitamin C” refers to ascorbic acid and its derivatives such as, for example, sodium ascorbyl phosphate, ascorbyl tetraisopalmitate, 3-O-cetyl ascorbic acid and/or ascorbyl glucoside.

The matrix is rich in unsaturated and polyunsaturated fatty acids, which provide a high affinity towards ROS, and therefore, allows the capsule to selectively convey to skin areas rich in ROS the antioxidant agents incorporated in it, which act as “scavengers”, neutralizing the ROS.

In an embodiment of the invention, the at least one antioxidant is in an amount from greater than or equal to 5% to less than or equal to 20 by weight of the total weight of the capsule.

The matrix according to the invention further comprises one or more excipients selected from surfactants, suspending agents, humectants, and their mixtures from greater than or equal to 10% to less than or equal to 12% by weight of the total weight of the capsule. Examples of excipients are polysorbates, polyacrylates, xanthan gum, alkyl glucosides and glycols.

Furthermore, the capsules of the invention can further comprise one or more active ingredients incorporated in said matrix, wherein the one or more active ingredients are selected from the group consisting of vitamins, peptides, minerals, growth factors and natural extracts.

The aforementioned active ingredients have the function of restoring the physiological balance, repairing and stimulating the autogenous repair mechanisms of the skin damaged by excessive concentrations of ROS.

In an embodiment of the invention, a method is disclosed for the making capsules to be incorporated into a skin care product. This method comprises the steps of melting a solid lipophilic part at approximately 50±5 degrees Celsius, obtaining a melted lipophilic part, solubilizing or dispersing in water: one or more water-soluble active ingredients, at least one lipophilic component rich in unsaturated fatty acids, at least one surfactant and/or suspension agent and/or humectant, at least one antioxidant agent, obtaining an aqueous solution or suspension, cooling said aqueous solution or suspension to a temperature lower than 10 degrees Celsius, obtaining a cooled aqueous solution or suspension, combining said melted lipophilic part with said cooled aqueous solution or suspension, under stable agitation, obtaining an emulsion, screening said emulsion through a mesh with holes with a size on the order of 0.1-10 μm, obtaining a microemulsion, composed of said at least one capsule, drying said microemulsion in a vacuum at a controlled temperature lower than 40 degrees Celsius and/or adding preservatives to the microemulsion.

An alternate embodiment of the invention is another method for making the capsules. The method comprises the steps of (i) melting the lipid component; (ii) dispersing the one or more unsaturated fatty acids in the melted lipid component of step (i); (iii) dispersing at least one antioxidant agent and the one or more excipients in water previously heated to a temperature from greater than or equal to 40 degrees Celsius to less than or equal to 50 degrees Celsius; (iv) adding said dispersion obtained in step (ii) to said aqueous dispersion obtained in step (iii), under stirring, obtaining an emulsion; (v) cooling the emulsion obtained in step (iv) to a temperature from greater than or equal to 5 degrees Celsius to less than or equal to 15 degrees Celsius, obtaining an emulsion comprising capsules in an aqueous phase.

An example of “stable agitation” is constant stirring between 100 rpm and 1000 rpm.

Examples of preservatives are sodium benzoate, potassium sorbate, isothiazolinones, phenoxyethanol, formaldehyde eliminators, or chelators.

The capsules can then be incorporated into a serum, cream, or lotion to be functionalized by mixing them with this serum, cream, or lotion at a temperature below 40 degrees Celsius, at low speed, for a maximum of 10 minutes.

An example of “low speed” is a rotation speed between 100 rpm and 200 rpm.

After incorporating these capsules into the serum, cream, or lotion, the serum, cream, or lotion containing the capsules can be packaged in a hermetically-sealed container to avoid exposure to air and avoid formation of ROS within the product.

The matrices of the capsules and the capsules themselves have an ORAC greater than 500 μmol/100 g.

In this way, the capsules have a greater affinity to ROS than the products existing today.

In an alternate embodiment, the present invention relates to a process for the preparation of capsules according to the invention, comprising the steps of melting the lipid component; dispersing the one or more unsaturated fatty acids in the melted lipid component; dispersing the at least one antioxidant agent and the one or more excipients in water previously heated to a temperature from greater than or equal to 40 degrees Celsius to less than or equal to 50 degrees Celsius; combining the dispersion of water, antioxidant agent, and excipient with the dispersion of the lipid component, and the unsaturated fatty acid, under stirring, to form an emulsion; and cooling the emulsion to a temperature from greater than 5 degrees Celsius to less than 15 degrees Celsius, to form an emulsion of capsules in an aqueous phase.

In one embodiment, the stirring in step (iv) is carried out at a constant speed between 1000 and 8000 revolutions per minute.

In an alternative embodiment, before step (v) the emulsion obtained in step (iv) is sieved through a net with holes ranging in size from greater than or equal to 0.1 to less than or equal to 50 μm, obtaining an emulsion consisting of capsules in an aqueous phase with a size ranging from greater than or equal to 0.1 to less than or equal to 50 μm. Said emulsion, after cooling in step (v), can then be dried under vacuum at a temperature below 40 degrees Celsius and/or stabilized by adding preservatives such as, for example, sodium benzoate, potassium sorbate, isothiazolinones, phenoxyethanol, formaldehyde eliminators, chelating agents.

In an embodiment of the process according to the invention, the lipid component is a mixture of at least two of mimosa wax (Acacia decurrens), jojoba seed wax, sunflower seed wax, polyglycerol, pentaerythrityl distearate, transesterification products of polyglycerol 3 and at least one wax chosen from jojoba wax, mimosa wax and sunflower seed wax. The choice of the above described mixture of waxes advantageously allows the capsules to be prepared with a process that does not require high temperatures or organic solvents. Therefore, in one embodiment, organic solvents are not used.

The capsules of the invention obtained as described above can then be incorporated into a skin care product selected from the group consisting of a serum, a cream and a lotion, mixing the capsules with said skin care product at a temperature of less than or equal to 40 degrees Celsius and a speed between 100 and 200 revolutions per minute until complete homogenization.

The present invention also relates to a skin care product comprising the capsules according to the invention. Said product may be a serum, a cream, or a lotion, optionally packaged in an airtight container to protect the skin care product from the formation of ROS.

Skin conditions associated with ROS accumulation include premature aging, loss of elasticity, loss of hydration, hyperpigmentation, dyschromia, acne, and imbalance of physiological balance.

The present invention also refers to capsules for use in the topical treatment of a condition associated with an accumulation of ROS in the skin of a person, wherein each capsule comprises a matrix comprising a lipid component; one or more unsaturated fatty acids; one or more excipients selected from surfactants, suspending agents, humectants and their mixtures; and at least antioxidant agent incorporated in said matrix.

The present invention also relates to a method for topical treatment of a condition associated with an accumulation of ROS in the skin of a person, by applying one or more capsules or a skin care product described herein to an area of skin of a person. In an embodiment of this invention, the method for topical treatment comprises applications of a capsule, or skin care product containing one or more capsules, at least once per week for a plurality of weeks.

The following is a non-limiting example of the preparation of capsules of the invention.

To prepare 1 kg of a composition comprising the capsules of the invention, to be used as an intermediate in the preparation of cosmetic, or skin care, products, the procedure was as follows:

(i) 90 g of a blend of jojoba wax, mimosa wax and sunflower seed wax (ACTICIRE® MB, ACTICIRE-GATTE FOSSE) and 30 g of a blend of natural polyunsaturated fatty acids obtained from safflower oil (Vitamin F forte, CLR Berlin) were melted in a beaker by heating at a temperature of 55 degrees Celsius under magnetic stirring at 200 rpm for 10 minutes until the phase was completely melted;

(ii) 30 g of a mixture of natural polyunsaturated fatty acids obtained from safflower oil (Vitamin F forte, CLR Berlin) were added to the melted lipid mixture obtained in step (i), under magnetic stirring at 200 rpm while maintaining the temperature at 55 degrees Celsius, until a homogeneous “fat” melted mixture is obtained;

(iii) an aqueous dispersion was prepared by adding to 758 ml of water heated to 45 degrees Celsius subsequently under stirring (500 rpm for 10 minutes):
20 g of decyl glucoside,
100 g of sodium ascorbyl phosphate,
2 g of hyaluronic acid;

(iv) the aqueous dispersion prepared in point (iii) was added to the fat mixture prepared in point (ii) by maintaining the temperature at 48 degrees Celsius under stirring at 3500 rpm for 10 minutes, obtaining an emulsion of capsules in a aqueous phase;

(iv-a) the emulsion obtained in step (iv) was sieved through a net with pores with a diameter of 25 μm, obtaining an emulsion of capsules with a diameter of up to 25 μm in an aqueous phase;

(v) the emulsion obtained in step (iv-a) was cooled to 10 degrees Celsius by means of a water bath to accelerate the solidification of the lipid part of the capsule;

Thus conceived, the invention is susceptible to numerous modifications and variants, all falling under the inventive concept; furthermore, all the details can be replaced by other technically equivalent elements. Further, features of the various disclosed embodiments of the invention can be combined and such combinations fall within the scope of the invention.

In practice, any materials may be used, so long as they are compatible with the specific use, as well as the contingent dimensions and forms, according to needs and the state of the art.

We claim:

1. A capsule, comprising:
a matrix comprising:
a lipid component having a melting temperature from greater than or equal to 40 degrees Celsius to less than or equal to 55 degrees Celsius, wherein the lipid component is from greater than or equal to 20 percent to less than or equal to 60 percent by weight of the total weight of the capsule;
an unsaturated fatty acid, wherein the unsaturated fatty acid is from greater than or equal to 20 percent to less than or equal to 30 percent by weight of the total weight of the capsule;
an excipient wherein the excipient is from greater than or equal to 10 percent to less than or equal to 12 percent by weight of the total weight of the capsule; and
an antioxidant agent incorporated in said matrix.

2. A capsule according to claim 1 wherein said lipid component comprises:
a first component selected from the group consisting of mimosa wax (Acacia decurrens), jojoba seed wax, sunflower seed wax;
a second component selected from the group consisting of mimosa wax (Acacia decurrens), jojoba seed wax, sunflower seed wax, polyglycerol, pentaerythrityl distearate, and transesterification products of polyglycerol; and
wherein the first component and the second component are different.

3. A capsule according to claim 1, wherein said matrix has an iodine index greater than or equal to 60 g Iodine/100 g of the matrix, wherein the iodine index is measured according to the ASTM D1959-97 standard.

4. A capsule according to claim 1, wherein the unsaturated fatty acid comprises a polyunsaturated fatty acid, and wherein the polyunsaturated fatty acid is at least 60 percent by weight of the total weight of the unsaturated fatty acid.

5. A capsule according to claim 1, wherein the antioxidant agent is from greater than or equal to 5 percent to less than or equal to 20 percent by weight of the total weight of the capsule.

6. A capsule according to claim 1, wherein said the unsaturated fatty acid is selected from the group consisting of C16-C24 fatty acids.

7. A capsules according to claim 1, wherein the excipient is selected from the group consisting of surfactants, suspending agents, and humectants.

8. A capsule according to claim 1, wherein the antioxidant agent is selected from the group consisting of BHT (butylhydroxytoluene), vitamin E, vitamin C, BHA (butylhydroxyanisole), polyphenols, carotenoids, superoxide dismutase, and bisulfites.

9. A capsule according to claim 1, further comprising an active ingredient incorporated in said matrix, wherein the active ingredient is selected from the group consisting of vitamins, peptides, minerals, growth factors, and natural extracts.

10. A process for the preparation of a capsule, comprising the steps of:
providing a lipid component having a melting temperature of greater than or equal to 40 degrees Celsius to less than or equal to 55 degrees Celsius;
melting the lipid component to form a melted lipid component;
dispersing an unsaturated fatty acid in the melted lipid component to form a dispersion of the lipid component and the unsaturated fatty acid;
dispersing an antioxidant agent and an excipient in water previously heated to a temperature from greater than or equal to 40 degrees Celsius to less than or equal to 50 degrees Celsius to form a dispersion of water, antioxidant agent, and excipient;
combining the dispersion of water, antioxidant agent, and excipient with the dispersion of the lipid component and the unsaturated fatty acid, under stirring, to form an emulsion; and
cooling the emulsion to a temperature from greater than 5 degrees Celsius to less than 15 degrees Celsius, to form an emulsion of capsules in an aqueous phase.

11. The process according to claim 10 wherein no organic solvent is used.

12. A method for topical treatment of a condition associated with an accumulation of Reactive Oxygen Species (ROS) in an area of skin of a person, comprising applying a capsule to the area of skin of the person, the capsule comprising:
a matrix comprising:
a lipid component having a melting temperature from greater than or equal to 40 degrees Celsius to less than or equal to 55 degrees Celsius, wherein the lipid component is from greater than or equal to 20 percent to less than or equal to 60 percent by weight of the total weight of the capsule;
an unsaturated fatty acid, wherein the unsaturated fatty acid is from greater than or equal to 20 percent to less than or equal to 30 percent by weight of the total weight of the capsule;
an excipient wherein the excipient is from greater than or equal to 10 percent to less than or equal to 12 percent by weight of the total weight of the capsule; and
an antioxidant agent incorporated in said matrix.

13. A skin care product comprising a capsule according to claim 1.

14. A method for the topical treatment of a condition associated with an accumulation of Reactive Oxygen Species (ROS) in an area of skin of a person comprising applying the skin care product of claim 13 to the area of skin of the person.
